(19) United States
(12) Patent Application Publication
(10) Pub. No.: US 2003/0049692 A1
(43) Pub. Date: Mar. 13, 2003

(54) Detection of anti-glycolipid antibodies by latex agglutination assay

(76) Inventors: Norman Latov, Irvington, NY (US); Armin Alaedini, New York, NY (US)

Correspondence Address: John P White, Cooper & Dunham, 1185 Avenue of the Americas, New York, NY, 10036, US

(21) Appl. No.: 10/088,775

(22) PCT Filed: Aug. 28, 2001
(86) PCT No.: PCT/US01/26708

(51) Int. Cl. G01N 33/53; G01N 33/542; C08B 37/00

(52) U.S. Cl. 435/7.9; 536/53

(57) ABSTRACT

The present invention comprises a method for detecting antiglycolipid autoantibodies in a subject who has or who may develop an autoimmune neuropathy. The present invention comprises a method for detecting antiganglioside autoantibodies in a subject. The present invention also provides methods for detecting multiple antiganglioside autoantibodies in a subject, simultaneously or consecutively. The present invention also provides methods for quantitating ganglioside autoantibodies in a subject. The present invention also provides a method of diagnosing autoimmune neuropathy in subjects with peripheral neuropathies. The present invention also provides a method of diagnosing autoimmune neuropathy in celiac disease in a subject.

This application is a continuation in part of U.S. Ser. No. 09/649,229 filed Aug. 28, 2000, the contents of which are hereby incorporated by reference into the subject application.

Throughout this application, various references are referred to within parentheses. Disclosures of these publications in their entireties are hereby incorporated by reference into this application to more fully describe the state of the art to which this invention pertains. Full bibliographic citation for these references may be found at the end of this application, preceding the claims.

### BACKGROUND OF THE INVENTION

Elevated levels of serum autoantibodies directed against gangliosides are closely associated with acute and chronic autoimmune neuropathies. For example, highly elevated titers of serum IgM anti-GM1 ganglioside antibodies are closely associated with multifocal motor neuropathy (reported to occur in 20% to 85% of patients with multifocal motor neuropathy or reversible lower motor neuron disease), but low titers are commonly present in normal individuals or other diseases. Antibodies to gangliosides are implicated in the pathogenesis of several autoimmune neuropathic syndromes, including the Guillain-Barré syndrome (1, 2), and a number of chronic peripheral neuropathies (3). These antibodies react with oligosaccharide determinants of major or minor gangliosides, which are highly concentrated in the peripheral nerves.

In several cases, the antibodies recognize oligosaccharide determinants that are shared by different gangliosides. For example, anti-GM1 ganglioside antibodies in motor neuropathy often react with the Gal(b1-3)GalNAc epitope which is shared by GD1b (4); antibodies to GD1b in sensory ataxic neuropathy recognize disialosyl epitopes shared by GD2, GD3, GT1b, and GQ1b (5, 6); antibodies to GD1a in motor dominant neuropathy recognize the NeuAc(a2-3)Gal(b1-3) moiety shared with GT1b and GM3 (7); and anti-GQ1b ganglioside antibodies in the Miller Fisher variant of the Guillain-Barré syndrome react with the disialosyl moiety which also characterizes GD3 and GD1b gangliosides among others (8).

Reflecting this, assays for the detection of anti-GM1 antibodies are therefore increasingly used in clinical practice to aid in the evaluation and diagnosis of patients suspected of having these diseases. At present, anti-glycolipid antibodies are routinely detected by ELISA, which measures serum antibody binding to purified individual glycolipids coated onto microwells (9). This assay system is relatively cumbersome, requires several days to perform, and takes place under non-physiologic conditions of temperature and serum dilution. In addition, routine testing is limited to single major gangliosides (and not multiple antibodies), and therefore may miss sera with antibodies that react with minor gangliosides, or with as yet uncharacterized gangliosides. Alternative liposome agglutination assays have proved difficult to manipulate in terms of consistency and reproducible assays, as well as having spontaneous agglutination problems which can give false-positives, and stability problems over time.

The present invention discloses an agglutination assay for antiganglioside autoantibody detection and also discloses that anti-ganglioside antibodies can be detected in samples from subjects presenting neuropathies in celiac disease which may serve as a basis for diagnosis. The new assay described herein can serve as a rapid and effective method for detecting, quantifying or screening for anti-ganglioside antibodies in patients with acute or chronic immune-mediated neuropathies or other disease producing antiganglioside autoantibodies. It would be particularly useful for detecting antibodies that react with minor, or as yet uncharacterized gangliosides, or with epitopes shared by several different gangliosides. Further, this invention discloses a method for detecting multiple antiglycolipid antibodies simultaneously, or rapidly detecting single antibodies that bind to multiple gangliosides. A color coding method disclosed here allows titering of different antibodies simultaneously. The invention is considerably faster and more flexible than the ELISA method currently used.

### SUMMARY OF THE INVENTION

This invention provides a method of detecting the presence of an antibody directed against a ganglioside in a subject comprising:

(a) contacting a liquid sample from the subject with the ganglioside, such ganglioside being affixed to at least two separate solid particles, under conditions permitting the antibody if present in the sample to form a complex with the ganglioside, which complex comprises such solid particles; and

(b) detecting the presence of any complex formed in step (a), wherein the presence of such complexes indicates the presence of the antibody in the subject.

This invention also provides a method of detecting in a subject the presence of at least two different antibodies, each of which antibodies is directed against a different type of ganglioside comprising:

(a) contacting a liquid sample from the subject with one such type of ganglioside, such ganglioside being affixed to at least two separate solid particles, under conditions permitting the antibody directed against said type of ganglioside if present in the sample to form a complex with the ganglioside, which complex comprises such solid particles;

(b) contacting such liquid sample with a different type of ganglioside, such different type of ganglioside being affixed to at least two separate solid particles, under conditions permitting the antibody directed against such different type of ganglioside if present in the sample to form a complex with such different type of ganglioside, which complex comprises such solid particles; and

(c) detecting the presence of any complex formed in step (b) and any complex formed in step (c), wherein the presence of complexes formed in both step (b) and step (c) indicates the presence in the subject of such different antibodies.

This invention further provides the instant method, wherein steps (a) and (b) are performed simultaneously.

This invention further provides the instant method, wherein the solid particles having affixed thereto said one such type of ganglioside are the same color and the solid particles having affixed thereto said different type of ganglioside are of a different color.

This invention further provides the instant methods, wherein the antibody is directed against more than one ganglioside.

This invention further provides the instant methods, wherein the antibody is directed against one ganglioside.

This invention also provides a method of quantitating the amount of an antibody directed against a ganglioside present in a subject comprising:

(a) contacting a plurality of identical liquid samples from the subject with the ganglioside, each such sample comprising the ganglioside affixed to at least two separate solid particles, such particles having affixed thereto a predetermined amount of such ganglioside, wherein the predetermined amount used to contact each said sample is different, under conditions permitting the antibody if present in the sample to form a complex with the ganglioside, which complex comprises such solid particles; and

(b) detecting the presence in each such sample of any complex formed in step (a), and correlating such detection of complexes in each such sample with a predefined reference standard indicative of the amount of the antibody present in the subject so as to quantitate the amount of the antibody present in the subject.

This invention also provides a method of quantitating the amount of an antibody directed against a ganglioside present in a subject comprising:

(a) contacting a plurality of liquid samples from the subject with the ganglioside, each such sample being differently diluted and such ganglioside being affixed to at least two separate solid particles, such particles having affixed thereto a predetermined amount of such ganglioside, wherein the predetermined amount used to contact each said sample is the same, under conditions permitting the antibody if present in the sample to form a complex with the ganglioside, which complex comprises such solid particles; and

(b) detecting the presence in each such sample of any complex formed in step (a), and correlating such detection of complexes in each such sample with a predefined reference standard indicative of the amount of the antibody present in the subject so as to quantitate the amount of the antibody present in the subject.

This invention further provides the instant methods, wherein the liquid sample is human sera.

This invention further provides the instant methods, wherein the liquid sample is chosen from the group consisting of plasma, saliva, tears, mucosal discharge, urine, peritoneal fluid, cerebrospinal fluid, lymphatic fluid, bone marrow, tissue, lymph nodes or culture media.

This invention further provides the instant methods, wherein the solid particles comprise polystyrene latex.

This invention further provides the instant methods, wherein the solid particles comprise carbonsol.

This invention further provides the instant methods, wherein the ganglioside is covalently affixed to the solid particles.

This invention further provides the instant methods, wherein the ganglioside is chosen from the group consisting of GM1, GM2, GM3, GD1, GD2, GD3, GD1a, GD1b, GT1b or GQ1b.

This invention further provides the instant methods, wherein the ganglioside comprises total brain ganglioside extract. This invention further provides the instant method, wherein the source of the extract is a bovid.

This invention further provides the instant methods, wherein the ganglioside comprises tissue ganglioside extract.

This invention further provides the instant methods, wherein the antiganglioside antibody is an autoantibody.

This invention further provides the instant methods, wherein the antiganglioside antibody is chosen from the group consisting of anti-GM1, anti-GM2, anti-GM3, anti-GD1, anti-GD2, anti-GD3, anti-GD1a, anti-GD1b, anti-GT1b or anti-GQ1b.

This invention further provides a method of diagnosing whether a subject has autoimmune neuropathy, comprising quantitating the amount of an antibody directed against a ganglioside in the subject using either of the instant methods, wherein the presence of a predefined amount of the antibody indicates that the subject is suffering from autoimmune neuropathy.

This invention further provides the instant method, wherein the neuropathy is Guillain-Barré syndrome.

This invention further provides the instant method, wherein the neuropathy is a Guillain-Barré syndrome variant.

This invention further provides the instant method, wherein the neuropathy is a peripheral neuropathic disease.

This invention further provides the instant method, wherein the neuropathy is a multifocal motor neuropathy.

This invention further provides a method of diagnosing whether a subject that has Celiac disease suffers from autoimmune neuropathy, comprising quantitating the amount of an antibody directed against a ganglioside in the subject using either of the instant methods, wherein the presence of a predefined amount of the antibody indicates that the subject is suffering from autoimmune neuropathy.

This invention further provides the instant method, wherein the antibody is directed against GM1.

This invention further provides the instant method, wherein the antibody is directed against GD1a.

This invention further provides a method of determining if a subject is predisposed to become afflicted with an autoimmune neuropathy, comprising quantitating the amount of an antibody directed against a ganglioside in the subject using either of the instant methods, wherein the presence of a predefined amount of the antibody indicates that the subject is predisposed to become afflicted with an autoimmune neuropathy.

This invention further provides the instant method, wherein the neuropathy is Guillain-Barré syndrome.

This invention further provides the instant method, wherein the neuropathy is a Guillain-Barré syndrome variant.

This invention further provides the instant method, wherein the neuropathy is a peripheral neuropathic disease.

This invention further provides the instant method, wherein the neuropathy is a multifocal motor neuropathy.

This invention further provides a method of determining if a subject with Celiac disease is predisposed to become afflicted with an autoimmune neuropathy, comprising quantitating the amount of an antibody directed against a ganglioside in the subject using either of the instant methods, wherein the presence of a predefined amount of the antibody indicates that the subject is predisposed to become afflicted with an autoimmune neuropathy.

This invention further provides the instant method, wherein the antibody is directed against GM1.

This invention further provides the instant method, wherein the antibody is directed against GD1a.

### BRIEF DESCRIPTION OF THE FIGURES

FIG. 1: Analysis of patient sera with latex agglutination assay and ELISA.

FIG. 2: Comparison of ELISA and latex agglutination assay in detection of anti-GM1 antibodies in sera of patients with MMN.

FIG. 3: Latex agglutination assay in detection of anti-GM1 antibodies in sera of patients with MMN using latex particles coated with different ratios of GM1 to GD1a.

FIG. 4: Analysis of patient sera with ELISA and latex agglutination assay.

FIG. 5: Comparison of ELISA and latex agglutination assay for antiganglioside antibody-positive sera.

### DETAILED DESCRIPTION OF THE INVENTION

This invention provides a method of detecting the presence of an antibody directed against a ganglioside in a subject comprising:

(a) contacting a liquid sample from the subject with the ganglioside, such ganglioside being affixed to at least two separate solid particles, under conditions permitting the antibody if present in the sample to form a complex with the ganglioside, which complex comprises such solid particles; and

(b) detecting the presence of any complex formed in step (a), wherein the presence of such complexes indicates the presence of the antibody in the subject.

Solid particles are generally constructed of unreactive material and are of consistent size, for example 0.3 μm diameter latex polystyrene beads. Two separate particles having ganglioside there affixed can be bound by an antibody. In one embodiment ganglioside is covalently affixed to the microparticles. In a different embodiment the ganglioside is not covalently affixed to the microparticle. In one embodiment microparticles comprise polystyrene latex. In one embodiment the microparticles comprise carbonsol.

The subject includes, but is not limited to, a human, a primate, a mouse, a rat, a guinea pig or a rabbit. In a preferred embodiment the subject is a human.

In different embodiments the ganglioside is chosen from the group consisting of GM1, GM2, GM3, GD1, GD2, GD3, GD1a, GD1b, GT1b or GQ1b, where G=ganglioside. In another embodiment the ganglioside comprises total brain ganglioside extract. In a further embodiment the source of the extract is a bovid. In one embodiment the ganglioside comprises tissue ganglioside extract.

In one embodiment the antiganglioside antibody is an autoantibody. In differing embodiments the antiganglioside antibody is chosen from the group consisting of anti-GM1, anti-GM2, anti-GM3, anti-GD1, anti-GD2, anti-GD3, anti-GD1a, anti-GD1b, anti-GT1b or anti-GQ1b, where G=ganglioside, e.g. anti-GM1 is an antibody directed against GM-1. The terms ‘antiganglioside antibody’ and ‘antibody directed against a ganglioside’ are used interchangeably.

In one embodiment the sample is human sera. In differing embodiments the sample is chosen from the group consisting of plasma, saliva, tears, mucosal discharge, urine, peritoneal fluid, cerebrospinal fluid, lymphatic fluid, bone marrow, tissue, lymph nodes or culture media. This invention also provides a method of detecting in a subject the presence of at least two different antibodies, each of which antibodies is directed against a different type of ganglioside comprising:

(a) contacting a liquid sample from the subject with one such type of ganglioside, such ganglioside being affixed to at least two separate solid particles, under conditions permitting the antibody directed against said type of ganglioside if present in the sample to form a complex with the ganglioside, which complex comprises such solid particles;

(b) contacting such liquid sample with a different type of ganglioside, such different type of ganglioside being affixed to at least two separate solid particles, under conditions permitting the antibody directed against such different type of ganglioside if present in the sample to form a complex with such different type of ganglioside, which complex comprises such solid particles; and

(c) detecting the presence of any complex formed in step (b) and any complex formed in step (c), wherein the presence of complexes formed in both step (b) and step (c) indicates the presence in the subject of such different antibodies.

This invention further provides the instant method, wherein steps (a) and (b) are performed simultaneously.

This invention further provides the instant method, wherein the solid particles having affixed thereto said one such type of ganglioside are the same color and the solid particles having affixed thereto said different type of ganglioside are of a different color.

Solid particles are generally constructed of unreactive material and are of consistent size, for example 0.3 μm diameter latex polystyrene beads. In one embodiment ganglioside is covalently affixed to the microparticles.

In a different embodiment the ganglioside is not covalently affixed to the microparticle. In one embodiment microparticles comprise polystyrene latex. In one embodiment the microparticles comprise carbonsol.

The subject includes, but is not limited to, a human, a primate, a mouse, a rat, a guinea pig or a rabbit. In a preferred embodiment the subject is a human.

In different embodiments the ganglioside is chosen from the group consisting of GM1, GM2, GM3, GD1, GD2, GD3, GD1a, GD1b, GT1b or GQ1b, where G=ganglioside. In another embodiment the ganglioside comprises total brain ganglioside extract. In a further embodiment the source of the extract is a bovid. In one embodiment the ganglioside comprises tissue ganglioside extract.

In one embodiment the antiganglioside antibody is an autoantibody. In differing embodiments the antiganglioside antibody is chosen from the group consisting of anti-GM1, anti-GM2, anti-GM3, anti-GD1, anti-GD2, anti-GD3, anti-GD1a, anti-GD1b, anti-GT1b or anti-GQ1b, where G=ganglioside as described hereinabove. The terms ‘antiganglioside antibody’ and ‘antibody directed against a ganglioside’ are used interchangeably.

In one embodiment the sample is human sera. In differing embodiments the sample is chosen from the group consisting of plasma, saliva, tears, mucosal discharge, urine, peritoneal fluid, cerebrospinal fluid, lymphatic fluid, bone marrow, tissue, lymph nodes or culture media.

This invention further provides the instant methods, wherein the antibody is directed against more than one ganglioside.

This invention further provides the instant methods, wherein the antibody is directed against one ganglioside.

This invention also provides a method of quantitating the amount of an antibody directed against a ganglioside present in a subject comprising:

(a) contacting a plurality of identical liquid samples from the subject with the ganglioside, each such sample comprising the ganglioside affixed to at least two separate solid particles, such particles having affixed thereto a predetermined amount of such ganglioside, wherein the predetermined amount used to contact each said sample is different, under conditions permitting the antibody if present in the sample to form a complex with the ganglioside, which complex comprises such solid particles; and

(b) detecting the presence in each such sample of any complex formed in step (a), and correlating such detection of complexes in each such sample with a predefined reference standard indicative of the amount of the antibody present in the subject so as to quantitate the amount of the antibody present in the subject.

This invention also provides a method of quantitating the amount of an antibody directed against a ganglioside present in a subject comprising:

(a) contacting a plurality of liquid samples from the subject with the ganglioside, each such sample being differently diluted and such ganglioside being affixed to at least two separate solid particles, such particles having affixed thereto a predetermined amount of such ganglioside, wherein the predetermined amount used to contact each said sample is the same, under conditions permitting the antibody if present in the sample to form a complex with the ganglioside, which complex comprises such solid particles; and

(b) detecting the presence in each such sample of any complex formed in step (a), and correlating such detection of complexes in each such sample with a predefined reference standard indicative of the amount of the antibody present in the subject so as to quantitate the amount of the antibody present in the subject.

Solid particles are generally constructed of unreactive material and are of consistent size, for example 0.3 μm diameter latex polystyrene beads. In one embodiment ganglioside is covalently affixed to the microparticles. In a different embodiment the ganglioside is not covalently affixed to the microparticle. In one embodiment microparticles comprise polystyrene latex. In one embodiment the microparticles comprise carbonsol.

The subject includes, but is not limited to, a human, a primate, a mouse, a rat, a guinea pig or a rabbit. In a preferred embodiment the subject is a human.

In different embodiments the ganglioside is chosen from the group consisting of GM1, GM2, GM3, GD1, GD2, GD3, GD1a, GD1b, GT1b or GQ1b, where G=ganglioside. In another embodiment the ganglioside comprises total brain ganglioside extract. In a further embodiment the source of the extract is a bovid. In one embodiment the ganglioside comprises tissue ganglioside extract.

In one embodiment the antiganglioside antibody is an autoantibody. In differing embodiments the antiganglioside antibody is chosen from the group consisting of anti-GM1, anti-GM2, anti-GM3, anti-GD1, anti-GD2, anti-GD3, anti-GD1a, anti-GD1b, anti-GT1b or anti-GQ1b, where G=ganglioside. The terms ‘antiganglioside antibody’ and ‘antibody directed against a ganglioside’ are used interchangeably.

In one embodiment the sample is human sera. In differing embodiments the sample is chosen from the group consisting of plasma, saliva, tears, mucosal discharge, urine, peritoneal fluid, cerebrospinal fluid, lymphatic fluid, bone marrow, tissue, lymph nodes or culture media.

This invention further provides a method of diagnosing whether a subject has autoimmune neuropathy, comprising quantitating the amount of an antibody directed against a ganglioside in the subject using the instant methods, wherein the presence of a predefined amount of the antibody indicates that the subject is suffering from autoimmune neuropathy. In one embodiment the neuropathy is Guillain-Barré syndrome. In another embodiment the neuropathy is a Guillain-Barré syndrome variant. Examples of Guillain-Barré syndrome variant include, but are not limited to, acute inflammatory demyelinating polyneuropathy, acute motor axonal neuropathy, Miller Fisher syndrome and acute motor and sensory axonal neuropathy. In one embodiment the neuropathy is a peripheral neuropathic disease. In one embodiment the neuropathy is a multifocal motor neuropathy.

This invention further provides a method of diagnosing whether a subject that has Celiac disease suffers from autoimmune neuropathy, comprising quantitating the amount of an antibody directed against a ganglioside in the subject using the instant method, wherein the presence of a predefined amount of the antibody indicates that the subject is suffering from autoimmune neuropathy. In one embodiment the antibody is directed against GM1. In one embodiment the antibody is directed against GD1a.

This invention further provides a method of determining if a subject is predisposed to become afflicted with an autoimmune neuropathy, comprising quantitating the amount of an antibody directed against a ganglioside in the subject using either of the instant methods, wherein the presence of a predefined amount of the antibody indicates that the subject is predisposed to become afflicted with an autoimmune neuropathy. In one embodiment the neuropathy is Guillain-Barré syndrome. In one embodiment the neuropathy is a Guillain-Barré syndrome variant. Examples of Guillain-Barré syndrome variant include, but are not limited to, acute inflammatory demyelinating polyneuropathy, acute motor axonal neuropathy, Miller Fisher syndrome and acute motor and sensory axonal neuropathy. In one embodiment the neuropathy is multifocal motor neuropathy. In one embodiment the neuropathic disease is a peripheral neuropathic disease.

This invention further provides a method of determining if a subject with Celiac disease is predisposed to become afflicted with an autoimmune neuropathy, comprising quantitating the amount of an antibody directed against a ganglioside in the subject using either of the instant methods, wherein the presence of a predefined amount of the antibody indicates that the subject is predisposed to become afflicted with an autoimmune neuropathy. In one embodiment the antibody is directed against GM1. In one embodiment the antibody is directed against GD1a. In one embodiment the subject is known to have Celiac disease. In another embodiment the subject is not known to have Celiac disease.

This invention will be better understood by reference to the Experimental Details which follow, but those skilled in the art will readily appreciate that the specific experiments detailed are only illustrative of the invention as described more fully in the claims which follow thereafter.

Experimental Details

First Series of Experiments

Materials and Methods

Serum Samples

Serum samples were obtained from 29 patients; eight with multifocal motor neuropathy (MMN), ten with chronic inflammatory demyelinating polyneuropathy (CIDP), six with amyotrophic lateral sclerosis (ALS), four with demyelinating neuropathy associated with anti-myelin-associated glycoprotein (anti-MAG) antibodies, and one with Miller Fisher syndrome (MFS). In addition, sera from five normal subjects were evaluated as controls. All patient sera were prepared, aliquoted, and stored at −20° C.

Preparation of Latex Particles

Latex beads were coated with GM1 ganglioside by passive adsorption. A 400 mg/mL solution of GM1 ganglioside (Sigma Chemicals, St. Louis, Mo.) was prepared by combining 40 mL of a 5 mg/mL stock solution of GM1 in methanol with 210 mL of H₂O and 250 mL of 100 mM 2-(N-morpholino)ethanesulfonic acid (MES) buffer (pH 6.1). A 1% suspension of 0.3 m blue polystyrene latex particles (Seradyn Particle Technology, Indianapolis, Ind.) was prepared from the 2.5% stock suspension by adding H₂O Adsorption of GM1 to the beads was initiated by addition of microparticle suspension to the ganglioside solution, followed by gentle stirring for 4 hours at room temperature. The suspension was then incubated for 72 hours at 4° C. The particles were washed twice with a solution of 1% BSA in 25 mM MES buffer (pH 6.1) by centrifugation at 9,800×g and 4° C., and resuspended in the same solution. The coated beads were incubated for 48 hours at 4° C. before use. Control latex particles were prepared by coating them with GD1a ganglioside (Sigma Chemicals, St. Louis, Mo.) in place of GM1, following the same procedure.

To determine whether titers of anti-GM1 antibodies could be quantified by testing for reactivity with beads containing decreasing concentrations of GM1, sera were tested for agglutination using beads that were coated with varying concentrations of GM1 and GD1a. Preparation of the latex particles was the same as described for GM1, with the difference that increasing quantities of GD1a were used to replace GM1, effectively lowering the concentration of GM1 coated. The following concentrations of GM1 were examined: 100% GM1, 50% GM1, 12% GM1, 6% GM1, 1.5% GM1, 0.75% GM1, and 0% GM1.

Agglutination Reaction

On a 3-ring glass slide (Cel-Line, Newfield, N.J.), 4.5 mL aliquots of serum were placed. To each ring, 4.5 mL of the coated latex particles was added and mixed thoroughly with a plastic applicator. The slide was rocked gently for 30 to 40 seconds. Positive agglutination, characterized by blue clumps of beads, indicated the presence of anti-GM1 antibodies. Particle agglutination was more easily visualized when using colored latex beads instead of white beads. Strong results were clearly visible with the naked eye. Weak results could be visualized by holding the slide to a light source and observing for agglutination from underneath. To minimize inter-operator variability, all results were confirmed using a microscope (× 40 magnification). In the absence of agglutination, the reaction was considered to be negative. If agglutination were present, it was scored from 1 to 3 according to the degree of agglutination, where 1 denotes weak agglutination and 3 strong agglutination.

Enzyme-Linked Immunosorbent Assay (ELISA)

The presence of anti-GM1 IgM in sera was also measured by the commonly used enzyme-linked immunosorbent assay, following previously described procedure (11), with minor modification. Wells in 96-well round-bottom polystyrene microtiter plates (Becton Dickinson, Franklin Lakes, N.J.) were coated with 0.5 mg of GM1 in 100 mL of methanol. After evaporation of the methanol, the wells were blocked by incubation with 300 mL of 1% bovine serum albumin (BSA) in 10 mM phosphate-buffered saline (154 mM NaCl, pH 7.4) (PBS) for 4 hours at 4° C., and 100 mL of BSA/PBS-diluted patient or control serum was added to the wells. Wells coated with BSA instead of serum served as control. The plates were incubated overnight at 4° C. and then washed with the BSA/PBS solution. Antibody binding was detected by the addition of 100 mL peroxidase-conjugated goat anti-human IgM secondary antibody (ICN Biomedicals, Costa Mesa, Calif.) after 1:1000 dilution in BSA/PBS solution (a final concentration of 2.14 mg/mL) to each well, and incubation for 2 hours at 4° C. Plates were then washed and 100 mL of developing solution comprised of 27 mM citric acid, 50 mM Na₂HPO₄, 5.5 mM o-phenylenediamine, and 0.01% H₂O₂ (pH 5-5.5) was added to each well. The plates were incubated at room temperature for 30 minutes before measuring absorbance at 450 nm. The titer for each specimen was assigned as the highest dilution in which the absorbance reading was 0.1 units greater than in the corresponding BSA-coated wells. Sera with titers of 800 or lower were considered to be negative for the presence of clinically significant amounts of anti-GM1 antibodies, as such titers are also seen in normal subjects (10).

Results

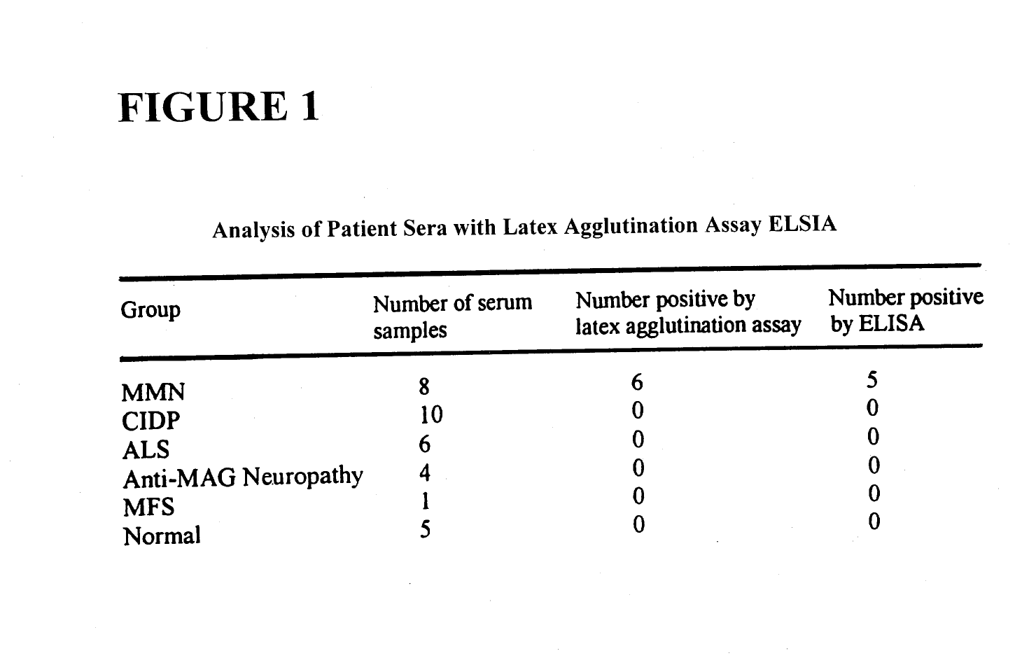

Sera from a total of 34 individuals were examined for anti-GM1 antibodies by both the agglutination assay and ELISA. Of the eight sera examined from MMN patients, six tested positive for anti-GM1 antibodies by the latex agglutination assay. A 11 sera from patients with CIDP, ALS, demyelinating neuropathy associated with anti-MAG antibodies, and MFS, as well as those from normal subjects were found to be negative (FIG. 1). All specimens were tested on at least three different occasions. The assay proved to have a high reproducibility as repeated tests on each serum gave identical results, with the rankings remaining the same.

Altering the concentration of coated GM1 antigen led to differences in reactivity with each serum. Undiluted sera with higher titers of anti-GM1 antibodies, as determined by ELISA, caused agglutination of microparticles coated with lower concentrations of antigen. The new agglutination assay was designed in such a manner as to give positive results only when testing sera with clinically significant titers of anti-GM1 antibodies. The sensitivity of the assay system was mainly dependent on the antigen concentration, that is the concentration of the coated GM1 ganglioside. That concentration was therefore adjusted to yield positive agglutination results with patient sera exhibiting anti-GM1 antibody titers of 800 or above, as measured in the ELISA system. Optimal results were obtained with incubation of a 1% suspension of 0.3 m latex beads with a 400 mg/mL solution of GM1.

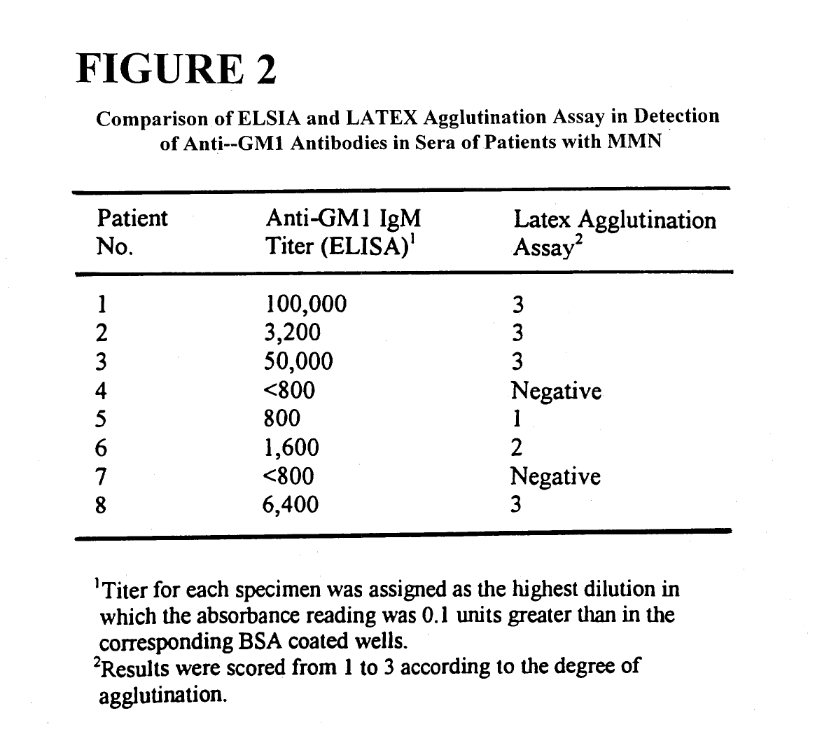

The agglutination assay exhibited equally good or better sensitivity when compared to the ELISA system. It gave positive results in all 5 of the 8 patients with MMN and elevated anti-GM1 antibodies as determined by ELISA, with titers ranging between 1,600 and 100,000 (FIG. 2). One other patient with MMN was positive by the agglutination assay but negative by ELISA, with a titer of 800. The two remaining patients with MMN were negative for anti-GM1 antibodies by both the agglutination and ELISA systems.

The agglutination assay appeared to be highly specific for patients with MMN, with none of the control patients or normal subjects exhibiting positive results. Four specimens with elevated levels of serum IgM and increased titers of anti-MAG antibodies, as well as a specimen from a patient with Miller Fisher syndrome (MFS) and antibodies against GQ1b ganglioside, tested negative for reactivity to GM1 with the agglutination assay.

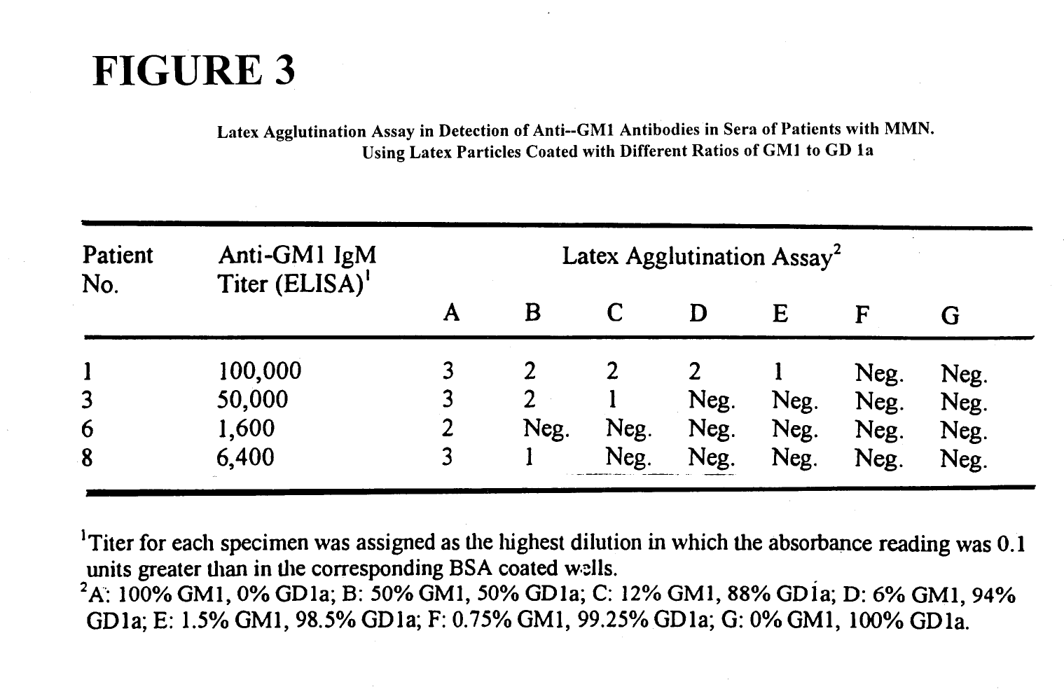

Four of the samples that exhibited reactivity to GM1 ganglioside in the agglutination assay were also tested for reactivity with latex particles coated with decreasing concentrations of GM1, in which GD1a was substituted (FIG. 3). None of the sera caused agglutination with particles coated with 100% GD1a, thus confirming the specificity of the GM1 reaction. On the other hand, all four sera yielded positive results with particles coated with less than 100% GM1; the higher the titer of anti-GM1 antibodies, the lower the concentration of the GM1 antigen that was required to produce agglutination. The serum with the highest concentration of anti-GM1 antibodies, having a titer of 100,000 by ELISA, reacted with beads that were coated with as little as 1.5% GM1.

Discussion

A novel latex agglutination assay was developed for detection of serum anti-GM1 antibodies. The assay detects a functional antibody-antigen interaction that results in agglutination and compares favorably to the ELISA system in sensitivity and specificity. Additional advantages of the new assay include substantial reduction in the cost and time required for performing the test. Unlike the ELISA, which takes two days to perform and requires a plate reader, the agglutination assay is completed in minutes and requires no special instruments.

The agglutination assay can be readily used to rapidly screen sera for the presence of anti-GM1 antibodies. In light of the fact that a large number of sera are negative for the presence of anti-GM1 antibodies, the assay aids in screening out negative serum samples. If information on antibody titer is desired, reactive sera can then be tested using the ELISA system, which measures antibody binding at increasing serum dilutions, or by the agglutination assay, which tests for reactivity using microparticles coated with decreasing antigen concentrations.

In addition to testing for antibodies to isolated glycolipids such as GM1, the agglutination assay could be useful in detecting antibody reactivities to one or more antigens in a mixture of glycolipids coated onto the latex particles. This could be used in the form of sensitive assays for detection of antibodies that react with shared epitopes on two or more glycolipids (14), or that recognize conformational epitopes that result from the interaction of two or more neighboring glycolipids (15). It could also be particularly useful in testing for the presence of antibodies directed against previously unrecognized antigenic glycolipids in other immune-mediated disorders.

### REFERENCES FOR FIRST SERIES OF EXPERIMENTS

1. Pestronk, A., Cornblath, D. R., Ilyas, A. A., et al., A treatable multifocal motor neuropathy with antibodies to GM1 ganglioside. Ann. Neurol. 1988; 24: 73-78.

2. Freddo, L., Yu, R. K., Latov, N., et al., Gangliosides GM1 and GD1b are antigens for IgM M-protein in a patient with motor neuron disease. Neurology. 1986; 36: 454-458.

3. Latov, N., Hays, A. P., Donofrio, P. D., et al., Monoclonal IgM with unique specificity to gangliosides GM1 and GD1b and to lacto-N-tetraose associated with human motor neuron disease. Neurology. 1988; 38: 763-768.

4. Kinsella, L. J., Lange, D. J., Trojaborg, W., Sadiq, S. A., Younger, D. S., and Latov, N., Clinical and electrophysiologic correlates of elevated anti-GM1 antibody titers. Neurology. 1994; 44: 1278-1282.

5. Taylor, B. V., Gross, L., and Windebank, A. J., The sensitivity and specificity of anti-GM1 antibody testing. Neurology. 1996; 47: 951-955.

6. Pestronk, A., and Choksi, R., Multifocal motor neuropathy: serum IgM anti-GM1 ganglioside antibodies in most patients detected using covalent linkage of GM1 to ELISA plates. Neurology. 1997; 49: 1289-1292.

7. Carpo, M., Allaria, S., Scarlato, G., and Nobile-Orazio, E., Marginally improved detection of GM1 antibodies by Covalink ELISA in multifocal motor neuropathy. Neurology. 1999; 53: 2206.

8. Marcus, D. M., Latov, N., Hsi, B. P., and Gillard, B. K., Measurement and significance of antibodies against GM1 ganglioside. Report of a workshop, Apr. 18, 1989, Chicago, Ill., USA. J. Neuroimmunol. 1989; 25: 255-259.

9. Holloway, R. G., and Feasby, T. E., To test or not to test? That is the question. Neurology. 1999; 53: 1905-1907.

10. Sadiq, S. A., Thomas, F. P., Kilidireas, K., et al., The spectrum of neurologic disease associated with anti-GM1 antibodies. Neurology. 1990; 40: 1067-1072.

11. Wirguin, I., Suturkova-Milosevic, L., Della-Latta, P., Fisher, T., Brown, R. H., and Latov, N., Monoclonal IgM antibodies to GM1 and asialo-GM1 in chronic neuropathies cross-react with Campylobacter jejuni lipopoly-saccharides. Ann. Neurol. 1994; 35: 698-703.

12. Kornberg, A. J., and Pestronk, A., Chronic motor neuropathies: diagnosis, therapy, and pathogenesis. Ann. Neurol. 1995; 37: S43-S50.

13. Marcus, D. M., Measurement and clinical importance of antibodies to glycosphingolipids. Ann. Neurol. 1990; 27: S53-S55.

14. Quarles, R. H., and Dalakas, M. C., Do anti-glycolipid antibodies cause human peripheral neuropathies? J. Clin. Invest. 1996; 97: 1136-1137.

15. Freddo, L., Hays, A. P., Nickerson, K. G., et al., Monoclonal anti-DNA IgM_{K} in neuropathy binds to myelin and to a conformational epitope formed by phosphatidic acid and gangliosides. J. Immunol. 1986; 137: 3821-3825.

Second Series of Experiments

Materials and Methods

Serum Samples

Serum samples were obtained from 45 patients: twelve with multifocal motor neuropathy (MMN), thirteen with Guillain-Barré syndrome (GBS), ten with chronic inflammatory demyelinating polyneuropathy (CIDP), six with amyotrophic lateral sclerosis (ALS), and four with demyelinating neuropathy associated with anti-myelin-associated glycoprotein (anti-MAG) antibodies. Criteria used for patient classification have been described before (11-14). In addition, serum samples from ten normal subjects were evaluated as controls. All patient sera were stored at −20° C.

Preparation of Latex Particles

Preparation of the microparticles was optimized particularly with regard to the amount of antigen coated on the surface of the particles, and the type of medium employed in the initiation of the reaction, such that normal sera would test negative in the final assay. Latex beads were coated with a total ganglioside preparation (Ca²⁺ salt) by passive adsorption. A 2 mg/mL solution of gangliosides (Sigma Chemicals, St. Louis, Mo.) was prepared by combining 105 mL of a 4.76 mg/mL stock solution of gangliosides in H₂O with 20 mL of methanol and 125 mL of 100 mM 2-(N-morpholino)ethanesulfonic acid (MES) buffer (pH 6.1). A 1% suspension of 0.3 m blue polystyrene latex particles (Seradyn Particle Technology, Indianapolis, Ind.) was prepared from the 2.5% stock suspension by adding H₂O. Adsorption of gangliosides to the beads was initiated by addition of 125 mL of microparticle suspension to the ganglioside solution, followed by gentle stirring for 4 hrs at room temperature. The suspension was then incubated for 72 hours at 4° C. The particles were washed twice with a solution of 1% bovine serum albumin (BSA) in 25 mM MES buffer (pH 6.1) by centrifugation at 9,800×g and 4° C., and resuspended in the same solution. The coated beads were incubated for 48 hrs at 4° C. before use.

Agglutination Reaction

On a 3-ring glass slide (Cel-Line, Newfield, N.J.), 5 mL aliquots of serum were placed. To each ring, 5 mL of the coated latex beads was added and mixed thoroughly with a plastic applicator. The slide was rocked gently for 30 to 40 seconds. Positive agglutination, characterized by blue clumps of beads, indicated the presence of anti-ganglioside antibodies. Colored latex beads were used instead of white beads because of the ease with which positive agglutination results could be visualized. Strong results were clearly visible with the naked eye. Weak results could be visualized by holding the slide to a light source, and observing for agglutination from underneath. In order to minimize inter-operator variability, all results were confirmed using a microscope (× 40 magnification). Results were scored from 1 to 3 according to the degree of agglutination, while in the absence of agglutination, the reaction was considered to be negative.

Enzyme-Linked Immunosorbent Assay (ELISA)

The presence of antibodies directed against GM1 and GQ1b in sera was determined by the enzyme-linked immunosorbent assay, following previously described procedure (15), with minor modification. Wells in 96-well round-bottom polystyrene microtiter plates (Becton Dickinson, Franklin Lakes, N.J.) were coated with 0.5 mg of the individual gangliosides (Sigma Chemicals, St. Louis, Mo.) in 100 mL of methanol. Wells to which only methanol was added served as controls. After evaporation of the methanol, all wells were blocked by incubation with 300 mL of 1% BSA in 10 mM phosphate-buffered saline (154 mM NaCl, pH 7.4) (PBS) for 4 hours at 4° C. The plates were incubated overnight at 4° C., and then washed with the BSA/PBS solution. This was followed by the addition of 100 mL of peroxidase-conjugated goat anti-human IgM or IgG secondary antibody (ICN Biomedicals, Costa Mesa, Calif.) after 1:1000 and 1:800 dilution respectively in BSA/PBS solution (a final concentration of 2.14 mg/mL for both antibodies) to each well, and incubation for 2 hours at 4° C. Plates were then washed as before and 100 mL of developing solution comprised of 27 mM citric acid, 50 mM Na₂HPO₄, 5.5 mM o-phenylenediamine, and 0.01% H₂O₂ (pH 55.5) was added to each well. The plates were incubated at room temperature for 30 min, before measuring absorbance at 450 nm. The titer for each specimen was assigned as the highest dilution in which the absorbance reading was 0.1 units greater than in the corresponding control well. Sera with titers of 800 or less were considered to be negative for the presence of clinically significant amounts of antibodies against GM1, as such titers are also seen in normal subjects (9, 10). Similarly, only sera with titers of 100 and above were considered positive for anti-GQ1b antibodies.

Results

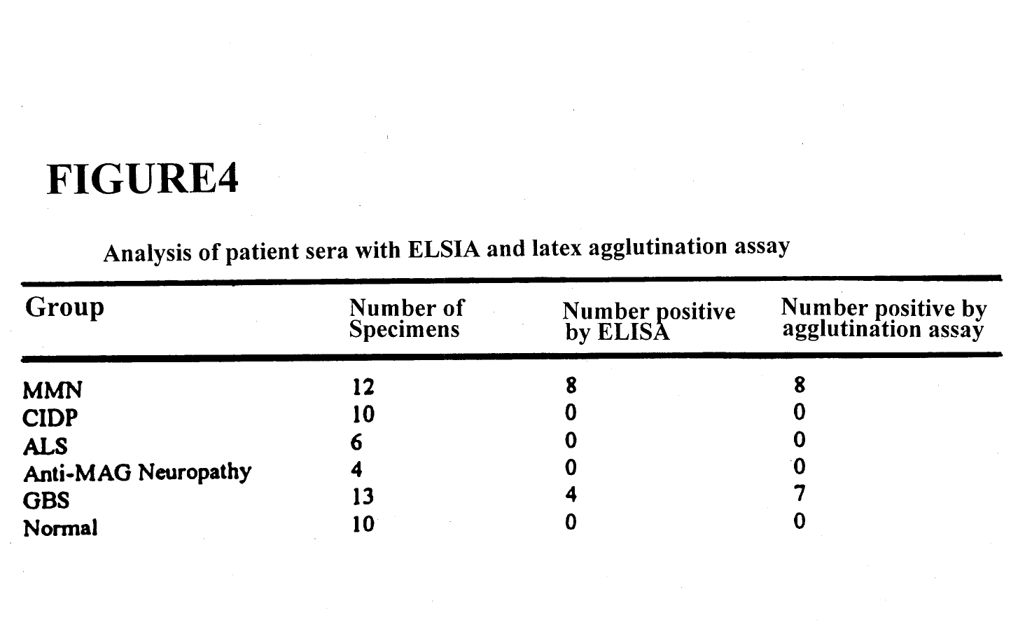

Sera from a total of 55 individuals were examined for anti-ganglioside antibodies by the agglutination immunoassay and ELISA. Of the twelve sera from MMN patients, eight were positive by both the agglutination assay (for anti-ganglioside antibodies), and the ELISA (for anti-GM1 antibodies). Of the thirteen sera from GBS patients, seven were positive for anti-ganglioside antibodies by the agglutination assay, while only four of these were positive for antibodies directed against GM1 or GQ1b by the ELISA system. All sera from patients with CIDP, ALS, and demyelinating neuropathy associated with MAG antibodies, in addition to those from normal subjects were found to be negative (FIG. 4). The new assay demonstrated high reproducibility as repeated tests on sera in a period of one week gave identical results, with the rankings staying the same.

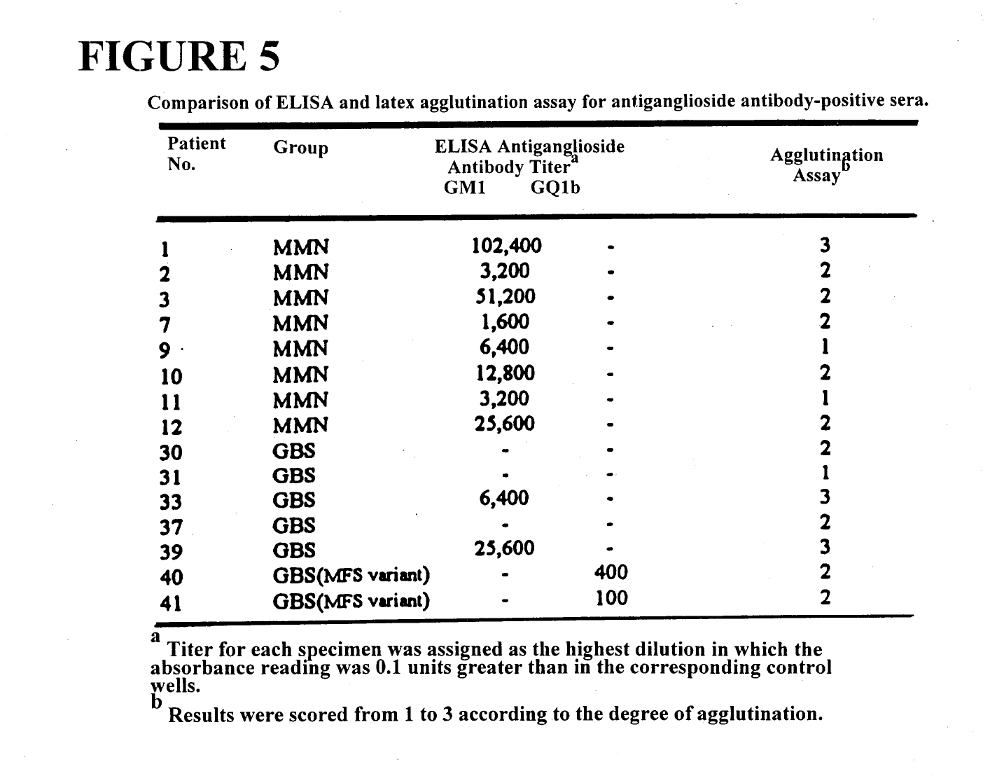

With regard to sera from patients with MMN where the antibody is directed against the GM1 ganglioside, the agglutination assay showed equally good sensitivity when compared to the ELISA system. It gave positive results in all 8 of the 12 patients with MMN and elevated titers of anti-GM1 antibodies as determined by ELISA, with titers ranging between 1,600 and 102,400 (FIG. 5). All serum samples from MMN patients with titers of 800 or less tested negative by the agglutination assay.

In analysis of sera from GBS patients, where the presence of several different anti-ganglioside antibody species have been reported, more patient sera were positive by the agglutination assay than the ELISA system. The two sera with elevated levels of IgG anti-GM1 antibodies and the two with elevated levels of IgG anti-GQ1b antibodies, with titers ranging from 100 to 25,600, as determined by ELISA, also tested positive with the agglutination assay. In addition, three other sera, which were found to be negative for antibodies against GM1 and GQ1b by ELISA, were positive for anti-ganglioside antibodies by the new agglutination assay. The remaining six serum samples were negative by both assays.

With the limited number of samples examined, the new assay demonstrated high specificity for patients with MMN and GBS, as none of the other patients or normal subjects exhibited positive results. Four sera with elevated levels of serum IgM and increased titers of anti-MAG antibodies tested negative for reactivity to gangliosides with the agglutination assay. Solutions of nonspecific human IgM and IgG in MES buffer (1 mg/mL) also yielded negative results when tested with the assay.

Multiple Antibody Detection

We tested sera for antibodies against multiple gangliosides in a single agglutination assay.

Materials and Methods

Sera from 256 patients with acute or chronic neuropathies, 6 patients with amyotrophic lateral sclerosis (ALS), and 10 normal subjects were tested for anti-ganglioside antibodies by the agglutination assay. Polystyrene microparticles were coated with a total ganglioside extract from bovine brain. When combined with serum, agglutination of microparticles signaled the presence of anti-ganglioside antibodies. Sera found to be positive by the agglutination assay were also tested by ELISA for IgM, IgG, and IgA antibodies to GM1, GM2, GD1a, GD1b, GQ1b, and GT1b gangliosides. Prior to the study, all sera were tested for anti-GM1 antibodies by ELISA.

Results

In the acute neuropathy group, 6 of 11 patients with Guillain-Barré Syndrome (GBS), 2 of 2 with Miller-Fisher Syndrome (MFS), and 1 with bilateral facial palsy were reactive by the ganglioside agglutination assay. When tested by ELISA, of the 6 GBS sera, 1 was positive for GM1, GM2, and GD1b, 1 for GM1 and GD1b, and 1 for GD1a alone, while 3 were unreactive. Sera from the 3 patients with MFS or bilateral facial palsy all reacted with GQ1b. In the chronic neuropathy group, 12 of 14 patients with multifocal motor neuropathy (MMN), and 5 of 214 patients with other types of neuropathy were positive by the new assay. In the ELISA system, of the 12 reactive MMN sera, 4 were positive for GM1 and GD1b, 3 for GM1 alone, 3 for GM1 and GM2, plus GD1a or GD1b, 1 for GM1, GD1b, and GQ1b, and 1 for GQ1b alone. Of the other 5 reactive sera, the ELISA system demonstrated binding to GM1 and GD1b in one, to GM1 alone in another, and no reactivity in 3. All 16 control sera were negative by the agglutination assay. All sera that were previously known to be positive for GM1 by the ELISA system were also positive by the new assay.

Discussion

These results show that the ganglioside agglutination system provides a rapid method for detecting antibodies to multiple gangliosides in a single assay. Sera that are positive by the agglutination assay, but negative by ELISA for the individual gangliosides tested, may recognize minor gangliosides or conformational epitopes which are not available in the ELISA system. The assay is useful for screening patients with suspected autoimmune neuropathies, particularly in situations where quick diagnosis is desired, as in the Guillain-Barré syndrome.

Also diagnosis of other autoimmune diseases presenting antiganglioside antibodies may be accelerated using this assay.

Titering by Sera Dilution

Instead of titering with antigens, titers can alternatively be performed using sera dilutions.

Materials and Methods

Such experiments were performed with the following agglutination reaction: On a 3-ring glass slide (Cel-Line, Newfield, N.J.), 5 mL aliquots of serum were placed. To each ring, 5 mL of the coated beads was added and mixed with a plastic applicator. The slide was rocked gently for 30 seconds. Positive agglutination, characterized by blue clumps of beads, indicated the presence of anti-ganglioside antibodies. Results were confirmed using a light microscope (× 40 magnification) and scored from 1 to 3 according to the degree of agglutination, where 1 denoted weak agglutination and 3 strong agglutination. In the absence of agglutination, the reaction was considered to be negative. Titration of sera was done only if the screening test was positive. Serial dilutions of sera were prepared in 10 mM phosphate-buffered saline (154 mM NaCl, pH 7.4) (PBS), in multiples of three. The titer for each specimen was assigned as the highest dilution in which the assigned score for the degree of agglutination was 1. All results were confirmed twice to reduce inter-operator variability.

Results

Sera was drawn from 112 individuals in this study. Sera were obtained from 40 patients with Guillain-Barré syndrome (GBS). Twenty eight of those in the GBS group were classified as acute inflammatory demyelinating polyneuropathy (AIDP), 7 as acute motor axonal neuropathy (AMAN), 1 as acute motor and sensory axonal neuropathy (AMSAN), and 4 as Miller Fisher syndrome (MFS). In addition, serum samples from 6 patients with amyotrophic lateral sclerosis (ALS), 20 patients with multiple sclerosis (MS), and 46 normal subjects were evaluated as controls. Standard ELISA tests were also performed.

Twenty one of the GBS patients (53%) were positive for anti-ganglioside antibodies by the agglutination immunoassay. Antibody titers ranged from 1 to 48. In comparison, 17 GBS patients (43%) showed elevated antibody levels when tested by ELISA for IgM and IgG antibodies against GM1, GM2, GD1a, GD1b, GT1b, and GQ1b, with titers ranging from 100 to 25,600. All samples that were positive by ELISA were also positive by the agglutination assay. No binding to GT1b was observed in any of the sera. For samples positive by both assays, antibody titers determined by sera dilution found with the agglutination assay showed correlation with those found by ELISA in most cases. All samples from patients with ALS or MS, or from normal subjects, were found to be negative by both assays. Among the 40 GBS sera, 12 of 28 from AIDP patients (43%), 5 of 7 from AMAN patients (71%), 3 of 4 from MFS patients (75%), and the one from the AMSAN patient, tested positive for anti-ganglioside antibodies by the agglutination assay.

Discussion

Measurement of serum anti-ganglioside autoantibody levels is increasingly used in the evaluation of patients with immune-mediated neuropathies. The currently available ELISA systems, however, are relatively time consuming and costly, and their use is limited due to issues of methodology, laboratory variability, and interpretation (16-20). Furthermore, in using these methods, testing against only a few standard gangliosides may miss some of the reactivities, whereas testing against every putative ganglioside antigen is inefficient and not always possible. In this study, a simple and quick agglutination assay capable of detecting a functional antibody-antigen interaction is described.

In patients with MMN, where the target antigen is the GM1 ganglioside, the new agglutination assay and ELISA yielded identical results. The degree of agglutination, however, was not found to correspond well to antibody titers as determined by ELISA, possibly due to differences in assay conditions. In contrast to the ELISA system, which measures binding of highly diluted serum at 4° C., the agglutination assay is performed under more physiologic elements of temperature and serum concentration, and measures a more functional interaction. The agglutination assay may thus better represent the antibody-antigen interaction that takes place in the human body.

In patients with GBS, the higher positivity rate for the agglutination assay (7/13) in comparison with ELISA (4/13) may be explained by the fact that the new assay detects the presence of all antiganglioside antibodies present in the serum, regardless of specificity or isotype. Sera from patients with GBS may cross react with or have antibodies to multiple gangliosides, including minor ones (21-23), and although most of the antibodies are IgG, antibodies of the IgM and IgA isotype have also been reported (24). We tested the sera against GM1 and GQ1b, which are the most common antigens described, but testing for all other gangliosides was beyond the scope of this study.

The new assay offers several advantages to the currently used ELISA system. It can detect the presence of antibodies to different gangliosides, while requiring only a few minutes to complete, and being more economical. It would be particularly useful in situations where rapid diagnosis and therapy are essential, as in the Guillain-Barré syndrome.

### REFERENCES FOR SECOND SERIES OF EXPERIMENTS

1. Asbury A K. New concepts of Guillain-Barré syndrome. J Child Neurol 2000;15:183-191.

2. Hughes R A C, Hadden R D M, Gregson N A, Smith K J. Pathogenesis of Guillain-Barré syndrome. J Neuroimmunol 1999;100:74-97.

3. Latov N. Pathogenesis and therapy of neuropathies associated with monoclonal gammopathies. Ann Neurol 1995;37(S1):S32-S42.

4. Nobile-Orazio E, Carpo M, Gename G, Meucci N, Sonnino S, Scarlato G. Anti-GM1 IgM antibodies in motor neuron disease and neuropathy. Neurology 1990;40:1747-1750.

5. Ilyas A A, Quarles R H, Dalakas M C, Fishman P H, Brady R O. Monoclonal IgM in a patient with paraproteinemic polyneuropathy binds to gangliosides containing disialosyl groups. Ann Neurol 1985;18:655-659.

6. Willison H J, Almemar A, Veitch J, Thrush D. Acute ataxic neuropathy with cross-reactive antibodies to GD1b and GD3 gangliosides. Neurology 1994;44:2395-2397.

7. Oga T, Kusunoki S, Fujimura H, Kuboki T, Yoshida T, Takai T. Severe motor-dominant neuropathy with IgM M-protein binding to the NeuAca2-3Ga1b-moiety. J Neurol Sci 1998;154:4-7.

8. Carpo M, Pedotti R, Lolli F, Pitrola A, Allaria S, Scarlato G, Nobile-Orazio E. Clinical correlate and fine specificity of anti-GQ1b antibodies in peripheral neuropathy. J Neurol Sci 1998;155:186-191.

9. Pestronk A. Motor neuropathies, motor neuron disorders, and antiglycolipid antibodies. [Review]. Muscle Nerve 1991;14:927-936.

10. Alaedini A, Latov N. Detection of anti-GM1 ganglioside antibodies in patients with neuropathy by a novel latex agglutination assay. J Immunoassay 2000 (In press).

11. Kinsella L J, Lange D J, Trojaborg W, Sadiq S A, Younger D S, Latov N. Clinical and electrophysiologic correlates of elevated anti-GM1 antibody titers. Neurology 1994;44:1278-1282.

12. Briani C, Brannagan T H 3^{rd}, Trojaborg W, Latov N,., Chronic inflammatory demyelinating polyneuropathy. Neuromuscul Disord 1996;6:311-325.

13. Van den Berg L, Hays A P, Nobile-Orazio E, Kinsella L J, Manfredini E, Corbo M, et al. Anti-MAG and anti-SGPG antibodies in neuropathy. Muscle Nerve 1996;19:637-643.

14. Asbury A K, Cornblath D R. Assessment of current diagnostic criteria for Guillain-Barré syndrome. Ann Neurol 1990;27:S21-S24.

15. Sadiq S A, Thomas F P, Kilidireas K, Protopsaitis S, Hays A P, Lee K W, et al. The spectrum of neurologic disease associated with anti-GM1 antibodies. Neurology 1990;40:1067-1072.

15. Marcus D M, Latov N, Hsi B P, Gillard B K. Measurement and significance of antibodies against GM1 ganglioside. Report of a workshop, Apr. 18, 1989, Chicago, Ill., USA. J Neuroimmunol 1989;25:255-259.

16. Carpo M, Allaria S, Scarlato G, Nobile-Orazio E. Marginally improved detection of GM1 antibodies by Covalink ELISA in multifocal motor neuropathy. [Technical brief]. Neurology 1999;53:2206.

17. Pestronk A. Testing for serum IgM binding to GM1 ganglioside in clinical practice. [Letter]. Neurology 2000;54:2353-2358.

18. Holloway R G, Feasby T E. To test or not to test? That is the question. [Editorial]. Neurology 1999;53:1905-1907.

19. Zielasek J, Ritter G, Magi S, Hartung H P, Toyka K V. A comparative trial of anti-glycoconjugate antibody assays: IgM antibodies to GM1. J Neurol 1994;241:475-480.

20. Ho T W, Willison H J, Nachamkin I, et al. Anti-GD1a antibody is associated with axonal but not demyelinating forms of Guillain-Barré syndrome. Ann Neurol 1999;45:168-173.

21. O'Leary C P, Veitch J, Durward W F, Thomas A M, Rees J H, Willison H J. Acute oropharyngeal palsy is associated with antibodies to GQ1b and GT1a gangliosides. J Neurol Neurosurg Psychiatry 1996;61:649-651.

22. Vriesendorp F J, Trigs W J, Mayer R F, Koski C L. Electrophysiological studies in Guillain-Barré syndrome: correlation with antibodies to GM1, GD1b and Campylobacter jejuni. J Neurol 1995;242:460-465.

23. Koga M, Yuki N, Takahashi M, Saito K, Hirata K. Close association of IgA anti-ganglioside antibodies with antecedent Campylobacter jejuni infection in Guillain-Barré and Fisher's syndromes. J Neuroimmunol 1998;81:138-143.

Third Series of Experiments

Celiac disease is an autoimmune gastrointestinal disorder, mediated by antibodies and T cells, which is provoked by ingestion of gluten proteins present in wheat, barley, and rye. It has been associated with peripheral neuropathy as well other neurological disorders. We analyzed sera from 20 patients with celiac disease for the presence of antiganglioside antibodies by the ganglioside agglutination immunoassay using microparticles coated with a total extract of bovine brain gangliosides. Controls can be taken from patients without celiac disease. Of the 20 sera tested, 5 were reactive by the agglutination assay. Of these 5 reactive sera, 4 were known to have peripheral neuropathy. When tested by ELISA for IgG, IgM, and IgA antibodies against GM1 and GD1a gangliosides, one serum was positive for IgG antibodies against GM1 and GD1a, one for IgG antibodies to GM1, and a third for IgG antibodies to GD1a. The two sera reactive by agglutination and negative by ELISA probably have antibodies to other, possibly minor gangliosides, or to conformation epitopes not detected by ELISA. The neuropathy associated with celiac disease appears to be associated with antiganglioside antibodies, which may contribute to the disease. The presence of IgG reactivity furthermore implicates a T cell-mediated response to ganglioside antigens.

What is claimed is:

1. A method of detecting the presence of an antibody directed against a ganglioside in a subject comprising:
(a) contacting a liquid sample from the subject with the ganglioside, such ganglioside being affixed to at least two separate solid particles, under conditions permitting the antibody if present in the sample to form a complex with the ganglioside, which complex comprises such solid particles; and
(b) detecting the presence of any complex formed in step (a), wherein the presence of such complexes indicates the presence of the antibody in the subject.

2. A method of detecting in a subject the presence of at least two different antibodies, each of which antibodies is directed against a different type of ganglioside comprising:
(a) contacting a liquid sample from the subject with one such type of ganglioside, such ganglioside being affixed to at least two separate solid particles, under conditions permitting the antibody directed against said type of ganglioside if present in the sample to form a complex with the ganglioside, which complex comprises such solid particles;
(b) contacting such liquid sample with a different type of ganglioside, such different type of ganglioside being affixed to at least two separate solid particles, under conditions permitting the antibody directed against such different type of ganglioside if present in the sample to form a complex with such different type of ganglioside, which complex comprises such solid particles; and
(c) detecting the presence of any complex formed in step (b) and any complex formed in step (c), wherein the presence of complexes formed in both step (b) and step (c) indicates the presence in the subject of such different antibodies.

3. The method of claim 2, wherein steps (a) and (b) are performed simultaneously.

4. The method of claim 2, wherein the solid particles having affixed thereto said one such type of ganglioside are the same color and the solid particles having affixed thereto said different type of ganglioside are of a different color.

5. The method of claim 1 or 2, wherein the antibody is directed against more than one ganglioside.

6. The method of claim 1 or 2, wherein the antibody is directed against one ganglioside.

7. A method of quantitating the amount of an antibody directed against a ganglioside present in a subject comprising:
(a) contacting a plurality of identical liquid samples from the subject with the ganglioside, each such sample comprising the ganglioside affixed to at least two separate solid particles, such particles having affixed thereto a predetermined amount of such ganglioside, wherein the predetermined amount used to contact each said sample is different, under conditions permitting the antibody if present in the sample to form a complex with the ganglioside, which complex comprises such solid particles; and
(b) detecting the presence in each such sample of any complex formed in step (a), and correlating such detection of complexes in each such sample with a predefined reference standard indicative of the amount of the antibody present in the subject so as to quantitate the amount of the antibody present in the subject.

8. A method of quantitating the amount of an antibody directed against a ganglioside present in a subject comprising:
(a) contacting a plurality of liquid samples from the subject with the ganglioside, each such sample being differently diluted and such ganglioside being affixed to at least two separate solid particles, such particles having affixed thereto a predetermined amount of such ganglioside, wherein the predetermined amount used to contact each said sample is the same, under conditions permitting the antibody if present in the sample to form a complex with the ganglioside, which complex comprises such solid particles; and
(b) detecting the presence in each such sample of any complex formed in step (a), and correlating such detection of complexes in each such sample with a predefined reference standard indicative of the amount of the antibody present in the subject so as to quantitate the amount of the antibody present in the subject.

9. The method of claim 1, 2, 7 or 8, wherein the liquid sample is human sera.

10. The method of claim 1, 2, 7 or 8, wherein the liquid sample is chosen from the group consisting of plasma, saliva, tears, mucosal discharge, urine, peritoneal fluid, cerebrospinal fluid, lymphatic fluid, bone marrow, tissue, lymph nodes or culture media.

11. The method of claim 1, 2, 7 or 8, wherein the solid particles comprise polystyrene latex.

12. The method of claim 1, 2, 7 or 8, wherein the solid particles comprise carbonsol.

13. The method of claim 1, 2, 7 or 8, wherein the ganglioside is covalently affixed to the solid particles.

14. The method of claim 1, 2, 7 or 8, wherein the ganglioside is chosen from the group consisting of GM1, GM2, GM3, GD1, GD2, GD3, GD1a, GD1b, GT1b or GQ1b.

15. The method of claim 1, 2, 7 or 8, wherein the ganglioside comprises total brain ganglioside extract.

16. The method of claim 15, wherein the source of the extract is a bovid.

17. The method of claim 1, 2, 7 or 8, wherein the ganglioside comprises tissue ganglioside extract.

18. The method of claim 1, 2, 7 or 8, wherein the antiganglioside antibody is an autoantibody.

19. The method of claim 1, 2, 7 or 8, wherein the antiganglioside antibody is chosen from the group consisting of anti-GM1, anti-GM2, anti-GM3, anti-GD1, anti-GD2, anti-GD3, anti-GD1a, anti-GD1b, anti-GT1b or anti-GQ1b.

20. A method of diagnosing whether a subject has autoimmune neuropathy, comprising quantitating the amount of an antibody directed against a ganglioside in the subject using the method of claim 7 or 8, wherein the presence of a predefined amount of the antibody indicates that the subject is suffering from autoimmune neuropathy.

21. A method of diagnosing whether a subject that has Celiac disease suffers from autoimmune neuropathy, comprising quantitating the amount of an antibody directed against a ganglioside in the subject using the method of claim 7 or 8, wherein the presence of a predefined amount of the antibody indicates that the subject is suffering from autoimmune neuropathy.

22. The method of claim 21, wherein the antibody is directed against GM1.

23. The method of claim 21, wherein the antibody is directed against GD1a.

24. The method of claim 19, wherein the neuropathy is Guillain-Barré syndrome.

25. The method of claim 19, wherein the neuropathy is a Guillain-Barré syndrome variant.

26. The method of claim 19, wherein the neuropathy is a peripheral neuropathic disease.

27. The method of claim 19, wherein the neuropathy is a multifocal motor neuropathy.

28. A method of determining if a subject is predisposed to become afflicted with an autoimmune neuropathy, comprising quantitating the amount of an antibody directed against a ganglioside in the subject using the method of claim 7 or 8, wherein the presence of a predefined amount of the antibody indicates that the subject is predisposed to become afflicted with an autoimmune neuropathy.

29. The method of claim 28, wherein the neuropathy is Guillain-Barré syndrome.

30. The method of claim 28, wherein the neuropathy is a Guillain-Barré syndrome variant.

31. The method of claim 28, wherein the neuropathy is a peripheral neuropathic disease.

32. The method of claim 28, wherein the neuropathy is a multifocal motor neuropathy.

33. A method of determining if a subject with Celiac disease is predisposed to become afflicted with an autoimmune neuropathy, comprising quantitating the amount of an antibody directed against a ganglioside in the subject using the method of claim 7 or 8, wherein the presence of a predefined amount of the antibody indicates that the subject is predisposed to become afflicted with an autoimmune neuropathy.

34. The method of claim 33, wherein the antibody is directed against GM1.

35. The method of claim 33, wherein the antibody is directed against GD1a.
